(19) United States
(12) Patent Application Publication
(10) Pub. No.: US 2014/0017182 A1
(43) Pub. Date: Jan. 16, 2014

(54) Topical Formulations Comprising DNA Repair Enzymes, and Methods of Use Thereof

(71) Applicant: Precision Dermatology, Inc., Cumberland, RI (US)

(72) Inventors: Mark W. Trumbore, Westford, MA (US); Drake D. Stimson, Terrace Park, OH (US); Paul Sowyrda, Medfield, MA (US)

(73) Assignee: Precision Dermatology, Inc., Cumberland, RI (US)

(21) Appl. No.: 13/937,605

(22) Filed: Jul. 9, 2013

Related U.S. Application Data
(60) Provisional application No. 61/670,836, filed on Jul. 12, 2012.

(51) Int. Cl. A61K 8/66; A61Q 17/04

(52) U.S. Cl. CPC A61K 8/66; A61Q 17/04
USPC 424/59

(57) ABSTRACT

Disclosed are methods of decreasing or preventing UV-induced skin damage, comprising the step of applying to an area of skin an effective amount of a topical formulation comprising a photolyase and an endonuclease. In certain embodiments, the formulation is applied before and after UV exposure.

### RELATED APPLICATIONS

This application claims the benefit of priority to U.S. Provisional Patent Application Ser. No. 61/670,836, filed Jul. 12, 2012.

### BACKGROUND OF THE INVENTION

Chronic excessive exposure to ultraviolet radiation (UVR) from sunlight is a causative factor in the development of photoaging and skin malignancies.¹⁻³ The harmful effects of UVR from sunlight are currently considered the major environmental risk factor for skin cancer and a complete carcinogen by damaging DNA⁴ and suppressing immune responses.⁵ The increased risk of cutaneous malignancies linked to chronic UVR exposure has been associated with direct DNA damage,⁶ which is mainly represented by the formation of cyclobutane pyrimidine dimers (CPD) that result from the photo [2+2] cycloaddition of the 5,6-double bond of two adjacent pyrimidine nucleotides.^{7,8}

Photolyase from A. nidulans^{9,10} and endonuclease from M. luteus^{11,12} are xenogenic DNA repair enzymes that can reverse the molecular events associated with skin aging and carcinogenosis caused by UVR exposure. In this regard, previous studies have shown that topical treatments utilizing photolyase and/or endonuclease in liposomal lotions may prove effective in reducing the risk of skin cancer in patients with defective DNA repair or in other at-risk patient populations by reversing the genome-damaging effects caused by UVR.^{9,10,13,14} Photolyases comprise efficient enzymes to remove the major UV-induced DNA lesion, i.e., CPD.¹⁵ The catalytic action of photolyase employs the light-driven injection of an electron onto the DNA lesion to trigger the cleavage of CPD.¹⁵ Similarly, endonuclease from M. Luteus acts as a CPD glycosylase/abasic lyase but—differently from photolyse—does not require light energy activation.¹²

Evidence suggests that specific molecular derangements—including telomere shortening¹⁶⁻¹⁹ and the upregulation of proto-oncogenes^{20,21}—may play a role in the setting of UVR-induced damage to biological tissues. Telomeres are specialized DNA, made up of a string of repeated TTAGGG located in the ends of chromosomes.²² They protect the ends of chromosomes from enzymatic degradation and shorten after each cell division.²² The shortening of telomere length has been suggested as a proxy for cellular senescence in the skin.¹⁶ Remarkably, Rochette et al.¹⁷ have reported that human telomeres are 7-fold hypersensitive to UVR-induced DNA damage compared with coding regions, and removal of CPDs in the telomere is almost absent. Although repeated UVR irradiations of diploid fibroblasts did not result in telomere shortening in vitro,¹⁷ little is known about the effects of repeated UVR irradiations on human telomeres in cells extracted from skin biopsies. Telomere length is also important in tumorigenesis. In this regard, Han et al.¹⁹ hypothesized that shorter telomere length in basal keratinocytes may trigger chromosomal aberrations that could then lead to the development of non-melanoma skin cancers. Skin exposure to UVR also results in a significant upregulation in the expression of the proto-oncogene c-FOS, one of the key transcription factors hyperexpressed in human cutaneous cancer.²³ Evidence has also suggested that cultured keratinocytes derived from photodamaged skin hyperexpress c-FOS,²⁴ which in turn may facilitate the development of skin cancer.

### SUMMARY OF THE INVENTION

In certain embodiments, the invention relates to the use of a photolyase and an endonuclease in the preparation of a topical formulation for the attenuation or prevention of UV-induced skin damage.

In certain embodiments, the invention relates to a topical formulation, comprising a photolyase; an endonuclease; and a dermatologically acceptable carrier or excipient.

In certain embodiments, the invention relates to a method of decreasing or preventing UV-induced skin damage, comprising the step of applying to an area of skin an effective amount of any one of the aforementioned topical formulations.

One aspect of the invention relates to the use of photolyase and endonuclease to prevent UV induced telomere shortening and c-fos transcription in skin. Another aspect of the invention relates to a method of preventing UV induced telomere shorting and c-fos expression in skin by topically applying photolyase and endonuclease. A third aspect of the present invention is a method of preventing UV induced telomere shortening and c-fos expression in skin by topically applying photolyase prior to sun exposure; and then applying endonuclease post exposure.

### BRIEF DESCRIPTION OF THE FIGURES

FIG. 1 depicts graphically that (i) repetitive UVR irradiations were significantly associated with a marked reduction in telomere length both in the UVR only positive control (mean T/S=1.10±0.09, P<0.001) and vehicle (placebo)+UVR sites (mean T/S=1.10±0.10, P<0.001) compared with the non-irradiated site (mean T/S=1.60±0.15); (ii) the application of a SS alone before each irradiation was associated with a statistically significant slightly longer telomere length in skin biopsies (mean T/S=1.20±0.09, P<0.05) as compared with the vehicle (placebo)+UVR sites; and (iii) the application of the SS before and endonuclease after each irradiation resulted in a mean T/S of 1.30±0.13 (P<0.01), which indicated that the telomere length was significantly higher than that observed at the site treated by the SS alone.

FIG. 2 tabulates the components and weight percentage of each component in an exemplary formulation.

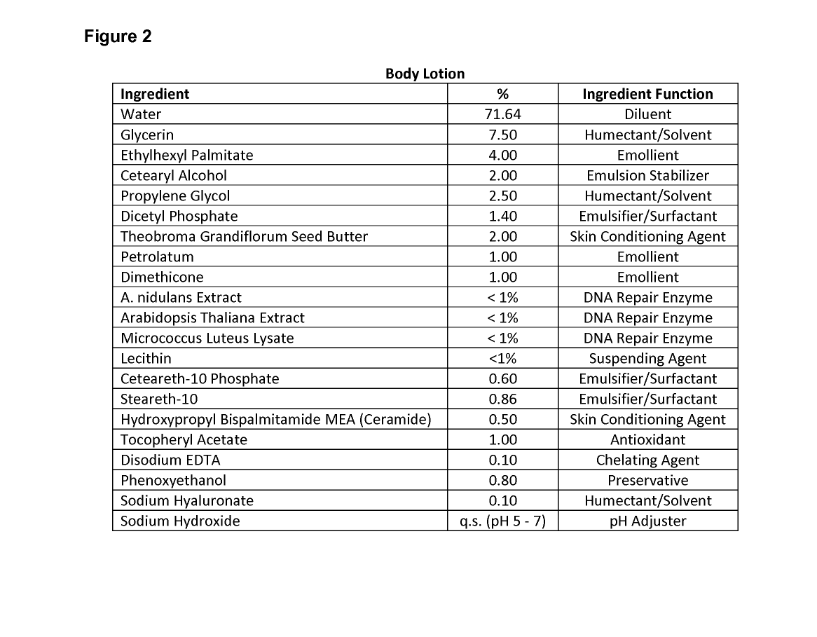

FIG. 3 tabulates the components and weight percentage of each component in an exemplary formulation.

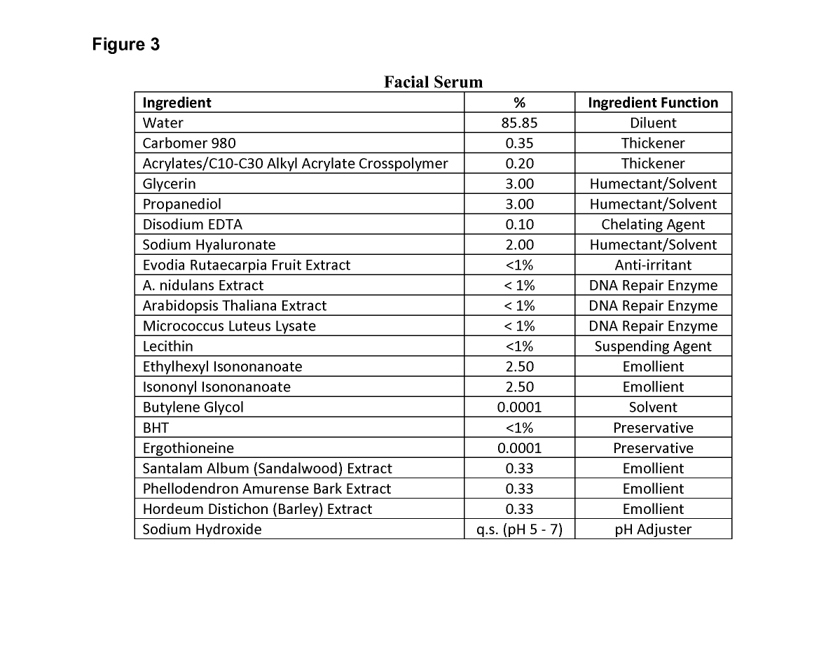

### DETAILED DESCRIPTION OF THE INVENTION

### Overview

Exposure to ultraviolet radiation (UVR) is one of the most important risk factors for skin aging and increases the risk of malignant transformation. Telomere shortening and an altered expression of the proto-oncogene c-FOS are among the key molecular mechanisms associated with photoaging and tumorigenesis. Photolyase from A. nidulans and endonuclease from M. luteus are xenogenic DNA repair enzymes which can reverse the molecular events associated with skin aging and carcinogenosis caused by UVR exposure. Remarkably, we have discovered that topical application of preparations containing DNA repair enzymes may prevent UVR-induced acute telomere shortening and FOS gene hyperexpression in human skin biopsies.

Twelve volunteers (Fitzpatrick skin types I and II) were enrolled for this experimental study, and six circular areas (10 mm diameter) were marked out on the non-exposed lower back of each participant. One site was left untreated (site 1: negative control), whereas the remaining five sites (designated sites 2-6) were exposed to solar-simulated UVR at 3 times the MED on four consecutive days. Site 2 received UVR only (site 2: positive control), whereas the following products were applied to sites 3-6, respectively: vehicle (moisturizer base cream; applied both 30 minutes before and immediately after each irradiation; site 3); a traditional sunscreen (SS, SPF 50) 30 minutes before irradiation and a vehicle immediately after irradiation (site 4); a SS 30 minutes before irradiation and an endonuclease preparation immediately after irradiation (site 5); and a SS plus photolyase 30 minutes before irradiation and an endonuclease preparation immediately after irradiation (site 6). Skin biopsies were taken 24 h after the last irradiation. The degrees of telomere shortening and c-FOS gene expression were measured in all specimens.

Remarkably, the T/S value of the site treated by SS plus photolyase 30 minutes before each irradiation and endonuclease immediately after irradiation was similar to that found in the non-irradiated skin (1.58±0.13), and was thus higher than that observed with the application of the SS before and endonuclease thereafter (P<0.001). In other words, the combined use of a SS plus photolyase 30 minutes before irradiation and an endonuclease preparation immediately after irradiation completely abrogated telomere shortening and c-FOS gene hyper-expression induced by the experimental irradiations. Remarkably, the topical application of preparations containing photolyase from A. nidulans and/or endonuclease from M. luteus appear to be clinically useful to prevent skin aging and carcinogenesis by abrogating UVR-induced telomere shortening and c-FOS gene hyper-expression.

### Discussion

The clinical results from this study demonstrate that the combined use of a SS plus photolyase thirty minutes before irradiation and an endonuclease preparation immediately after each UVR exposure completely abrogated telomere shortening and c-FOS gene hyperexpression induced by the experimental irradiations in humans. Taken together, our data suggest that the topical application of preparations containing DNA repair enzymes containing both a photolyase (e.g., from A. nidulans) and an endonuclease (e.g., from M. luteus) should be clinically useful to prevent skin aging and carcinogenesis by abrogating UVR-induced telomere shortening and c-FOS gene hyperexpression.

We are aware of no previous report detailing an investigation of the potential protective effects of SS against these two aging- and cancer-associated molecular alterations in the skin. An important finding of this study is the evidence of a limited protective ability of the SS alone toward the appearance of two highly sensitive age- and oncogenic-associated molecular alterations such as acute telomere shortening and c-FOS hyperexpression after repeated UVR irradiations of human skin. Although previous studies have shown that regular use of a broad-spectrum SS may be at least in part effective for preventing CPD formation in irradiated skin areas,^{9,27} the limited efficacy of SS for protecting telomeres against UVR-mediated damage is not surprising given that these parts of the chromosomes are hypersensitive to photodamage.¹⁷ Although repeated irradiations did not shorten telomere length in human fibroblasts cultured in vitro,¹⁷ our results clearly demonstrated that exposing human skin to UVR results in an acute telomere shortening. These apparent differences may be ascribed to the fact that in this study we measure telomere shortening in clinical skin biopsy samples, and not only in a specific skin cell type (fibroblasts) cultured in vitro.¹⁷ Pertinently, Ostoich et al.²⁸ have previously reported that the radiosensitivity and the transmission of radiation-induced telomere damage could largely differ in human fibroblasts compared with keratinocytes. Therefore, it can be hypothesized that the marked acute telomere-shortening effect of UVR in skin biopsies observed in this study could chiefly mirror keratinocyte-specific damage.

Previous studies have consistently shown that c-FOS is immediately induced in the skin upon UVR exposure and is increased in photodamaged skin.^{23,24} c-FOS is involved in a variety of physiological process including cell growth, differentiation, transformation, and signal transduction, and is one of the key transcription factor hyperexpressed in human non-melanoma skin cancers.²³ Our results indicate that topical application of xenogenic DNA repair enzymes on the human skin can greatly reduce the hyperexpression of c-FOS induced by UVR exposure. Because the combined application of photolyase and endonuclease did not result in any expression change of c-FOS at the mRNA level in human skin biopsies, even after repeated irradiations, we hypothesize that delivery of both enzymes combined with a SS to sun-exposed skin may potentially reduce the frequency of sunlight-associated non-melanoma skin malignancies.

Our clinical results support at the molecular level the efficacy of a combined UVR-protection strategy consisting of a traditional SS plus both photolyase and endonuclease for preventing two keys molecular derangements associated with skin aging and carcinogenesis (telomere shortening and c-FOS hyperexpression). Of note, these two molecular alterations could not be prevented by the use of a SS alone. In contrast, the optimal efficacy was seen when the both photolyase and endonuclease were applied together with the SS. A topical combination strategy consisting of a traditional SS plus two xenogenic DNA repair enzymes may be effective for restoring both telomere length and reducing UVR-induced c-FOS hyperexpression, which are paramount in the molecular pathogenesis of skin photoaging and non-melanoma cutaneous cancers in humans.

### DEFINITIONS

“Lysate” is a solution containing the contents of lysed cells. In certain embodiments, the term “lysate” and the term “extract” are synonymous. In other embodiments, the “extract” is the soluble portion of the lysate, after centrifugation and disposal of insoluble cellular matter, such as membrane fragments, vesicles, and nuclei. In certain embodiments, the extract comprises mostly cytosol.

“Lysis” is the breakage of the plasma membrane and optionally the cell wall of a biological organism sufficient to release at least some intracellular content, often by mechanical, viral or osmotic mechanisms that compromise its integrity.

“Lysing” is disrupting the cellular membrane and optionally the cell wall of a biological organism or cell sufficient to release at least some intracellular content.

In certain embodiments, the lysate or extract is isolated or extracted from a cell by lysing the cells. The step of lysing the cells can be achieved by any convenient means, including, but not limited to, heat-induced lysis, adding a base, adding an acid, using enzymes such as proteases and polysaccharide degradation enzymes such as amylases, using ultrasound, mechanical lysis (i.e., subjecting the biomass to pressure sufficient to lyse the cells, termed “pressing”), using osmotic shock, infection with a lytic virus, or expression of one or more lytic genes. Lysis is performed to release intracellular molecules which have been produced by the cell. Each of these methods for lysing cells can be used as a single method or in combination simultaneously or sequentially. The extent of cell disruption can be observed by microscopic analysis. Using one or more of the methods described herein, typically more than about 70% cell breakage is observed. In certain embodiments, cell breakage (lysis) is more than about 80% complete, more than about 90% complete, or about 100% complete.

In certain embodiments, the product is isolated or extracted from a cell by the methods described in US 2010/0151538, which is hereby incorporated by reference in its entirety.

In certain embodiments, the lysate or extract is isolated or extracted from a cell by adding a base to a suspension containing the cells. The base should be strong enough to hydrolyze at least a portion of the proteinaceous compounds of the cells. Bases which are useful for solubilizing proteins are known in the art of chemistry. Exemplary bases which are useful in the methods of the present invention include, but are not limited to, hydroxides, carbonates and bicarbonates of lithium, sodium, potassium, calcium, and mixtures thereof. In certain embodiments, the base is KOH. Base treatment of microalgae for cell disruption is described, for example, in U.S. Pat. No. 6,750,048, which is hereby incorporated by reference in its entirety.

In another embodiment of the present invention, the lysate or extract is isolated or extracted from a cell by using an enzyme. In certain embodiments, the enzymes for lysing a microorganism are proteases and polysaccharide-degrading enzymes such as hemicellulase, pectinase, cellulase, driselase, proteases, chymotrypsin, and proteinase K. Any combination of a protease and a polysaccharide-degrading enzyme can also be used.

In another embodiment, the lysate or extract is isolated or extracted from a cell using an expeller press. In this process, cells are forced through a screw-type device at high pressure, lysing the cells and causing the intracellular product to be released and separated from the protein and fiber (and other components) in the cell.

In another embodiment of the present invention, the lysate or extract is isolated or extracted from a cell by using ultrasound, i.e., sonication. Thus, cells can also by lysed with high frequency sound. The sound can be produced electronically and transported through a metallic tip to an appropriately concentrated cellular suspension. This sonication (or ultrasonication) disrupts cellular integrity based on the creation of cavities in cell suspension.

In another embodiment of the present invention, the lysate or extract is isolated or extracted from a cell by mechanical lysis. Cells can be lysed mechanically and optionally homogenized to facilitate collection of the product. For example, a pressure disrupter can be used to pump a cell containing slurry through a restricted orifice valve. High pressure (up to 1500 bar) is applied, followed by an instant expansion through an exiting nozzle. Cell disruption is accomplished by three different mechanisms: impingement on the valve, high liquid shear in the orifice, and sudden pressure drop upon discharge, causing an explosion of the cell. The method releases intracellular molecules. Alternatively, a ball mill can be used. In a ball mill, cells are agitated in suspension with small abrasive particles, such as beads. Cells break because of shear forces, grinding between beads, and collisions with beads. The beads disrupt the cells to release cellular contents. Cells can also be disrupted by shear forces, such as with the use of blending (such as with a high speed or Waring® blender), the french press, or even centrifugation in case of weak cell walls, to disrupt cells.

In another embodiment of the present invention, the lysate or extract is isolated or extracted from a cell by applying an osmotic shock.

In another embodiment of the present invention, the lysate or extract is isolated or extracted from a cell comprises infection of the cells with a lytic virus. A wide variety of viruses are known to lyse cells and are suitable for use in the present invention, and the selection and use of a particular lytic virus for a particular cell is within the level of skill in the art.

### Exemplary Constituents of Formulations of the Invention

Exemplary identities of various constituents of the topical formulations of the present invention are described below and in the Figures.

### 1. Vehicles, Solvents, and Diluents

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol, dimethicone, and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In one embodiment, formulations without methanol, ethanol, propanols, or butanols are desirable.

### 2. Surfactants and Emulsifiers

Many topical formulations contain chemical emulsions which use surface active ingredients (emulsifiers and surfactants) to disperse dissimilar chemicals in a particular solvent system. For example, most lipid-like (oily or fatty) or lipophilic ingredients do not uniformly disperse in aqueous solvents unless they are first combined with emulsifiers, which form microscopic aqueous soluble structures (droplets) that contain a lipophilic interior and a hydrophilic exterior, resulting in an oil-in-water emulsion. In order to be soluble in aqueous media, a molecule must be polar or charged so as to favorably interact with water molecules, which are also polar. Similarly, to dissolve an aqueous-soluble polar or charged ingredient in a largely lipid or oil-based solvent, an emulsifier is typically used which forms stable structures that contain the hydrophilic components in the interior of the structure while the exterior is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion droplet. Emulsion destabilization results in the aqueous and lipophilic ingredients separating into two layers, potentially destroying the commercial value of a topical product.

Surfactants suitable for use in the present invention may be ionic or non-ionic. These include, but are not limited to: cetyl alcohol, polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-10 phosphate, methylbenzethonium chloride, dicetyl phosphate, ceteth-10 phosphate (ceteth-10 is the polyethylene glycol ether of cetyl alcohol where n has an average value of 10; ceteth-10 phosphate is a mixture of phosphoric acid esters of ceteth-10), ceteth-20, Brij S10 (polyethylene glycol octadecyl ether, average Mₙ˜711), and Poloxamers (including, but not limited to, Poloxamer 188 (HO(C₂H₄O)ₐ(CH(CH₃)CH₂O)_{b}(C₂H₄O)ₐH, average molecular weight 8400) and Poloxamer 407 (HO(C₂H₄O)ₐ(CH(CH₃)CH₂O)_{b}(C₂H₄O)ₐH, wherein a is about 101 and b is about 56)). Appropriate combinations or mixtures of such surfactants may also be used according to the present invention.

Many of these surfactants may also serve as emulsifiers in formulations of the present invention.

Other suitable emulsifiers for use in the formulations of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol (cetearyl alcohol), ceteareth-12, ceteareth-20, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate, PEG-75 stearate, PEG-100 stearate, and PEG-100 stearate, ceramide 2, ceramide 3, stearic acid, cholesterol, steareth-2, and steareth-20, or combinations/mixtures thereof, as well as cationic emulsifiers like stearamidopropyl dimethylamine and behentrimonium methosulfate, or combinations/mixtures thereof.

### 3. Moisturizers, Emollients, and Humectants

One of the most important aspects of topical products in general, and cosmetic products in particular, is the consumer's perception of the aesthetic qualities of a product. For example, while white petrolatum is an excellent moisturizer and skin protectant, it is rarely used alone, especially on the face, because it is greasy, sticky, does not rub easily into the skin and may soil clothing. Consumers highly value products which are aesthetically elegant and have an acceptable tactile feel and performance on their skin.

Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800.

Suitable emollients or humectants for use in the formulations of the present invention include, but are not limited to, panthenol, cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, isononyl isononanoate, and 1,3-bis(N-2-(hydroxyethyl)palmitoylamino)-2-hydroxypropane.

In addition, appropriate combinations and mixtures of any of these moisturizing agents and emollients may be used in accordance with the present invention.

### 4. Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols, tocopheryl acetate, sodium ascorbate/ascorbic acid, ascorbyl palmitate, propyl gallate, and chelating agents like EDTA (e.g., disodium EDTA), citric acid, and sodium citrate.

In certain embodiments, the antioxidant or preservative comprises (3-(4-chlorophenoxy)-2-hydroxypropyl)carbamate.

In certain embodiments, antioxidants or preservatives of the present invention may also function as a moisturizer or emollient, for example.

In addition, combinations or mixtures of these preservatives or anti-oxidants may also be used in the formulations of the present invention.

### 5. Active Agents

The active agent may be any material that has a desired effect when applied topically to a mammal, particularly a human. Suitable classes of active agents include, but are not limited to, antibiotic agents, antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones. Mixtures of any of these active agents may also be employed. Additionally, dermatologically-acceptable salts and esters of any of these agents may be employed.

5.1 Antibiotics

Representative antibiotics include, without limitation, benzoyl peroxide, alfa terpineol, octopirox, erythromycin, zinc, tetracyclin, triclosan, azelaic acid and its derivatives, phenoxy ethanol and phenoxy propanol, ethyl acetate, clindamycin (e.g., clindamycin phosphate) and meclocycline; sebostats such as flavinoids; alpha and beta hydroxy acids; and bile salts such as scymnol sulfate and its derivatives, deoxycholate and cholate. The antibiotic can be an antifungal agent. Suitable antifungal agents include, but are not limited to, clotrimazole, econazole, ketoconazole, itraconazole, miconazole, oxiconazole, sulconazole, butenafine, naftifine, terbinafine, undecylinic acid, tolnaftate, and nystatin. Mixtures of these antibiotic agents may also be employed. Additionally, dermatologically-acceptable salts and esters of any of these agents may be employed.

5.2 Non-Steroidal Anti-Inflammatory Agents

Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac, fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone; and niacinamide. Mixtures of these non-steroidal anti-inflammatory agents may also be employed, as well as the dermatologically acceptable salts and esters of these agents. For example, etofenamiate, a flufenamic acid derivative, is particularly useful for topical application.

5.3 Steroidal Anti-Inflammatory Agents

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters (including betamethasone dipropionate), chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

5.4 Anesthetics

Suitable anesthetics include the aminoacylanilide compounds such as lidocaine, prilocalne, bupivacaine, levo-bupivacaine, ropivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, butamben, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine. Other suitable local anesthetics include ketocaine, dibucaine, amethocaine, propanacaine, and propipocaine.

5.5 Antimicrobial Agents

Suitable antimicrobial agents include, but are not limited to, antibacterial, antifungal, antiprotozoal and antiviral agents, such as beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin (e.g., clindamycin phosphate), ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, clindamycin phosphate, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, nystatin, tolnaftate, clotrimazole, anidulafungin, micafungin, voriconazole, lanoconazole, ciclopirox and mixtures thereof.

5.6 Keratolytic Agents

Suitable keratolytic agents include, but are not limited to, urea, salicylic acid, papain, sulfur, glycolic acid, pyruvic acid, resorcinol, N-acetylcysteine, retinoids such as retinoic acid (e.g., tretinoin) and its derivatives (e.g., cis and trans isomers, esters), retinol, alpha hydroxy acids, beta hydroxy acids, coal tar, and combinations thereof.

### 6. Buffer Salts

Suitable buffer salts are well-known in the art. Examples of suitable buffer salts include, but are not limited to sodium citrate, citric acid, sodium phosphate monobasic, sodium phosphate dibasic, sodium phosphate tribasic, potassium phosphate monobasic, potassium phosphate dibasic, and potassium phosphate tribasic.

### 7. Viscosity Modifiers

Suitable viscosity adjusting agents (i.e., thickening and thinning agents or viscosity modifying agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. In addition, appropriate combinations or mixtures of these viscosity adjusters may be utilized according to the present invention.

### 8. Additional Constituents

Additional constituents suitable for incorporation into the emulsions of the present invention include, but are not limited to: skin protectants, adsorbents, demulcents, emollients, moisturizers, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, lipids, immunomodulators, and pH adjusters (e.g., citric acid, sodium hydroxide, and sodium phosphate).

For example, lipids normally found in healthy skin (or their functional equivalents) may be incorporated into the emulsions of the present invention. In certain embodiments, the lipid is selected from the group consisting of ceramides, cholesterol, and free fatty acids. Examples of lipids include, but are not limited to, ceramide 1, ceramide 2, ceramide 3, ceramide 4, ceramide 5, ceramide 6, hydroxypropyl bispalmitamide MEA, and hydroxypropyl bislauramide MEA, and combinations thereof.

Examples of peptides that interact with protein structures of the dermal-epidermal junction include palmitoyl dipeptide-5 diaminobutyloyl hydroxythreonine and palmitoyl dipeptide-6 diaminohydroxybutyrate.

Examples of skin soothing agents include, but are not limited to algae extract, mugwort extract, stearyl glycyrrhetinate, bisabolol, allantoin, aloe, avocado oil, green tea extract, hops extract, chamomile extract, colloidal oatmeal, calamine, cucumber extract, and combinations thereof.

In certain embodiments, the compositions comprise bergamot or bergamot oil. Bergamot oil is a natural skin toner and detoxifier. In certain embodiments, it may prevent premature aging of skin and may have excellent effects on oily skin conditions and acne.

Examples of vitamins include, but are not limited to, vitamins A, D, E, K, and combinations thereof. Vitamin analogues are also contemplated; for example, the vitamin D analogues calcipotriene or calcipotriol.

In certain embodiments, the vitamin may be present as tetrahexyldecyl ascorbate. This compound exhibits anti-oxidant activity, inhibiting lipid peroxidation. In certain embodiments, use can mitigate the damaging effects of UV exposure. Studies have shown it to stimulate collagen production as well as clarifying and brightening the skin by inhibiting melanogenesis (the production of pigment) thereby promoting a more even skin tone.

Examples of sunscreens include, but are not limited to, p-aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide, 4-methylbenzylidene camphor, methylene bis-benzotriazolyl tetramethylbutylphenol, bis-ethylhexyloxyphenol methoxyphenyl triazine, terephthalylidene dicamphor sulfonic acid, drometrizole trisiloxane, disodium phenyl dibenzimidazole tetrasulfonate, diethylamino hydroxybenzoyl hexyl benzoate, octyl triazone, diethylhexyl butamido triazone, polysilicone-15, and combinations thereof.

Suitable fragrances and colors may be used in the formulations of the present invention. Examples of fragrances and colors suitable for use in topical products are known in the art.

Suitable immunomodulators include, but are not limited to, tetrachlorodecaoxide, deoxycholic acid, tacrolimus, pimecrolimus, and beta-glucan.

In certain embodiments, palmitoyl-lysyl-valyl-lysine bistrifluoroacetate is added. This peptide stimulates collagen synthesis in human fibroblasts.

Often, one constituent of a composition may accomplish several functions. In one embodiment, the present invention relates to constituents that may act as a lubricant, an emollient, or a skin-penetrating agent. In one embodiment, the multi-functional constituent is socetyl stearate, isopropyl isostearate, isopropyl palmitate, or isopropyl myristate.

### Exemplary Uses

In certain embodiments, the invention relates to the use of a photolyase and an endonuclease in the preparation of a topical formulation for the attenuation or prevention of UV-induced skin damage.

In certain embodiments, the invention relates to any one of the aforementioned uses, wherein the photolyase is from A. nidulans.

In certain embodiments, the invention relates to any one of the aforementioned uses, wherein the endonuclease is from M. luteus.

In certain embodiments, the invention relates to any one of the aforementioned uses, wherein the photolyase is from A. nidulans; and the endonuclease is from M. luteus.

In certain embodiments, the invention relates to the use of a photolyase in the preparation of a topical formulation for the attenuation or prevention of UV-induced skin damage.

In certain embodiments, the invention relates to any one of the aforementioned uses, wherein the photolyase is from A. nidulans.

In certain embodiments, the invention relates to the use of an endonuclease in the preparation of a topical formulation for the attenuation or prevention of UV-induced skin damage.

In certain embodiments, the invention relates to any one of the aforementioned uses, wherein the endonuclease is from M. luteus.

In certain embodiments, the invention relates to any one of the aforementioned uses, wherein the topical formulation comprises, consists essentially of, or consists of

| | % by weight of the |
|---|---|
| Ingredient | topical formulation |
| a diluent | from about 35 to about 95 |
| a humectant or a solvent | from about 5 to about 15 |
| a skin conditioning agent or an emollient | from about 4 to about 12 |
| a DNA repair enzyme | from about 1.5 to about 4.5 |
| a suspending agent | from about 0.1 to about 2 |
| an emulsion stabilizer, an emulsifier, or | from about 2.5 to about 7.5 |
| a surfactant | |
| an antioxidant | from about 0.5 to about 1.5 |
| a chelating agent | from about 0.05 to about 0.15 |
| a preservative | from about 0.4 to about 1.2 |
| a pH adjuster | |

In certain embodiments, the invention relates to any one of the aforementioned uses, wherein the formulation comprises, consists essentially of, or consists of

| | % by weight of the |
|---|---|
| Ingredient | topical formulation |
| Water | from about 35 to about 95 |
| Glycerin | from about 3 to about 11 |
| Ethylhexyl Palmitate | from about 2 to about 6 |
| Cetearyl Alcohol | from about 1 to about 3 |
| Propylene Glycol | from about 1 to about 4 |
| Dicetyl Phosphate | from about 0.5 to about 2.5 |
| Theobroma Grandiflorum Seed Butter | from about 1 to about 3 |
| Petrolatum | from about 0.5 to about 1.5 |
| Dimethicone | from about 0.5 to about 1.5 |
| A. nidulans Extract | from about 0.5 to about 1.5 |
| Arabidopsis Thaliana Extract | from about 0.5 to about 1.5 |
| Micrococcus Luteus Lysate | from about 0.5 to about 1.5 |
| Lecithin | from about 0.5 to about 1.5 |
| Ceteareth-10 Phosphate | from about 0.3 to about 0.9 |
| Steareth-10 | from about 0.3 to about 0.9 |
| Hydroxypropyl Bispalmitamide MEA | from about 0.2 to about 0.8 |
| (Ceramide) | |
| Tocopheryl Acetate | from about 0.5 to about 1.5 |
| Disodium EDTA | from about 0.05 to about 0.15 |
| Phenoxyethanol | from about 0.4 to about 1.2 |
| Sodium Hyaluronate | from about 0.05 to about 0.15 |
| Sodium Hydroxide | |

In certain embodiments, the invention relates to any one of the aforementioned uses, wherein the formulation comprises, consists essentially of, or consists of

| | % by weight of the |
|---|---|
| Ingredient | topical formulation |
| Water | about 71.64 |
| Glycerin | about 7.50 |
| Ethylhexyl Palmitate | about 4.00 |
| Cetearyl Alcohol | about 2.00 |
| Propylene Glycol | about 2.50 |
| Dicetyl Phosphate | about 1.40 |
| Theobroma Grandiflorum Seed Butter | about 2.00 |
| Petrolatum | about 1.00 |
| Dimethicone | about 1.00 |
| A. nidulans Extract | about 1 |
| Arabidopsis Thaliana Extract | about 1 |
| Micrococcus Luteus Lysate | about 1 |
| Lecithin | about 1 |
| Ceteareth-10 Phosphate | about 0.60 |
| Steareth-10 | about 0.86 |
| Hydroxypropyl Bispalmitamide MEA | about 0.50 |
| (Ceramide) | |
| Tocopheryl Acetate | about 1.00 |
| Disodium EDTA | about 0.10 |
| Phenoxyethanol | about 0.80 |
| Sodium Hyaluronate | about 0.10 |
| Sodium Hydroxide | |

In certain embodiments, the invention relates to any one of the aforementioned uses, wherein the topical formulation comprises, consists essentially of, or consists of

| | % by weight of the |
|---|---|
| Ingredient | topical formulation |
| a diluent | from about 45 to about 95 |
| a thickener | from about 0.25 to about 0.8 |
| a humectant or a solvent | from about 4 to about 12 |
| an anti-irritant | from about 0.5 to about 1.5 |
| a DNA repair enzyme | from about 1.5 to about 4.5 |
| a suspending agent | from about 0.5 to about 1.5 |
| an emollient | from about 3 to about 9 |
| a chelating agent | from about 0.05 to about 0.15 |
| a preservative | from about 0.5 to about 1.5 |
| pH Adjuster | |

In certain embodiments, the invention relates to any one of the aforementioned uses, wherein the topical formulation comprises, consists essentially of, or consists of

| | % by weight of the |
|---|---|
| Ingredient | topical formulation |
| Water | from about 45 to about 95 |
| Carbomer 980 | from about 0.1 to about 0.6 |
| Acrylates/C10-C30 Alkyl Acrylate | from about 0.1 to about 0.3 |
| Crosspolymer | |
| Glycerin | from about 1.5 to about 4.5 |
| Propanediol | from about 1.5 to about 4.5 |
| Disodium EDTA | from about 0.05 to about 0.15 |
| Sodium Hyaluronate | from about 1 to about 3 |
| Evodia Rutaecarpia Fruit Extract | from about 0.5 to about 1.5 |
| A. nidulans Extract | from about 0.5 to about 1.5 |
| Arabidopsis Thaliana Extract | from about 0.5 to about 1.5 |
| Micrococcus Luteus Lysate | from about 0.5 to about 1.5 |
| Lecithin | from about 0.5 to about 1.5 |
| Ethylhexyl Isononanoate | from about 2.50 |
| Isononyl Isononanoate | from about 2.50 |
| Butylene Glycol | from about 0.0001 |
| BHT | from about <1% |
| Ergothioneine | from about 0.0001 |
| Santalam Album (Sandalwood) Extract | from about 0.33 |
| Phellodendron Amurense Bark Extract | from about 0.33 |
| Hordeum Distichon (Barley) Extract | from about 0.33 |
| Sodium Hydroxide | |

In certain embodiments, the invention relates to any one of the aforementioned uses, wherein the topical formulation comprises, consists essentially of, or consists of

| | %, by weight of the |
|---|---|
| Ingredient | topical formulation |
| Water | about 85.85 |
| Carbomer 980 | about 0.35 |
| Acrylates/C10-C30 Alkyl Acrylate | about 0.20 |
| Crosspolymer | |
| Glycerin | about 3.00 |
| Propanediol | about 3.00 |
| Disodium EDTA | about 0.10 |
| Sodium Hyaluronate | about 2.00 |
| Evodia Rutaecarpia Fruit Extract | about 1 |
| A. nidulans Extract | about 1 |
| Arabidopsis Thaliana Extract | about 1 |
| Micrococcus Luteus Lysate | about 1 |
| Lecithin | about 1 |
| Ethylhexyl Isononanoate | about 2.50 |
| Isononyl Isononanoate | about 2.50 |
| Butylene Glycol | about 0.0001 |
| Butylated hydroxy toluene (BHT) | about 1 |
| Ergothioneine | about 0.0001 |
| Santalam Album (Sandalwood) Extract | about 0.33 |
| Phellodendron Amurense Bark Extract | about 0.33 |
| Hordeum Distichon (Barley) Extract | about 0.33 |
| Sodium Hydroxide | |

### Exemplary Formulations

In certain embodiments, the invention relates to a topical formulation, comprising a photolyase; an endonuclease; and a dermatologically acceptable carrier or excipient.

In certain embodiments, the invention relates to a topical formulation, consisting essentially of a photolyase; an endonuclease; and a dermatologically acceptable carrier or excipient.

In certain embodiments, the invention relates to a topical formulation, consisting of a photolyase; an endonuclease; and a dermatologically acceptable carrier or excipient.

In certain embodiments, the invention relates to any one of the aforementioned topical formulations, wherein the photolyase is from A. nidulans.

In certain embodiments, the invention relates to any one of the aforementioned topical formulations, wherein the endonuclease is from M. luteus.

In certain embodiments, the invention relates to any one of the aforementioned topical formulations, wherein the photolyase is from A. nidulans; and the endonuclease is from M. luteus.

In certain embodiments, the invention relates to a topical formulation, comprising a photolyase; and a dermatologically acceptable carrier or excipient.

In certain embodiments, the invention relates to a topical formulation, consisting essentially of a photolyase; and a dermatologically acceptable carrier or excipient.

In certain embodiments, the invention relates to a topical formulation, consisting of a photolyase; and a dermatologically acceptable carrier or excipient.

In certain embodiments, the invention relates to any one of the aforementioned topical formulations, wherein the photolyase is from A. nidulans.

In certain embodiments, the invention relates to a topical formulation, comprising an endonuclease; and a dermatologically acceptable carrier or excipient.

In certain embodiments, the invention relates to a topical formulation, consisting essentially of an endonuclease; and a dermatologically acceptable carrier or excipient.

In certain embodiments, the invention relates to a topical formulation, consisting of an endonuclease; and a dermatologically acceptable carrier or excipient.

In certain embodiments, the invention relates to any one of the aforementioned topical formulations, wherein the endonuclease is from M. luteus.

In certain embodiments, the invention relates to any one of the aforementioned topical formulations, wherein the topical formulation comprises, consists essentially of, or consists of

| | % by weight of the |
|---|---|
| Ingredient | topical formulation |
| a diluent | from about 35 to about 95 |
| a humectant or a solvent | from about 5 to about 15 |
| a skin conditioning agent or an emollient | from about 4 to about 12 |
| a DNA repair enzyme | from about 1.5 to about 4.5 |
| a suspending agent | from about 0.1 to about 2 |
| an emulsion stabilizer, an emulsifier, or | from about 2.5 to about 7.5 |
| a surfactant | |
| an antioxidant | from about 0.5 to about 1.5 |
| a chelating agent | from about 0.05 to about 0.15 |
| a preservative | from about 0.4 to about 1.2 |
| a pH adjuster | |

In certain embodiments, the invention relates to any one of the aforementioned topical formulations, wherein the formulation comprises, consists essentially of, or consists of

| | % by weight of the |
|---|---|
| Ingredient | topical formulation |
| Water | from about 35 to about 95 |
| Glycerin | from about 3 to about 11 |
| Ethylhexyl Palmitate | from about 2 to about 6 |
| Cetearyl Alcohol | from about 1 to about 3 |
| Propylene Glycol | from about 1 to about 4 |
| Dicetyl Phosphate | from about 0.5 to about 2.5 |
| Theobroma Grandiflorum Seed Butter | from about 1 to about 3 |
| Petrolatum | from about 0.5 to about 1.5 |
| Dimethicone | from about 0.5 to about 1.5 |
| A. nidulans Extract | from about 0.5 to about 1.5 |
| Arabidopsis Thaliana Extract | from about 0.5 to about 1.5 |
| Micrococcus Luteus Lysate | from about 0.5 to about 1.5 |
| Lecithin | from about 0.5 to about 1.5 |
| Ceteareth-10 Phosphate | from about 0.3 to about 0.9 |
| Steareth-10 | from about 0.3 to about 0.9 |
| Hydroxypropyl Bispalmitamide MEA | from about 0.2 to about 0.8 |
| (Ceramide) | |
| Tocopheryl Acetate | from about 0.5 to about 1.5 |
| Disodium EDTA | from about 0.05 to about 0.15 |
| Phenoxyethanol | from about 0.4 to about 1.2 |
| Sodium Hyaluronate | from about 0.05 to about 0.15 |
| Sodium Hydroxide | |

In certain embodiments, the invention relates to any one of the aforementioned topical formulations, wherein the formulation comprises, consists essentially of, or consists of

| | % by weight of the |
|---|---|
| Ingredient | topical formulation |
| Water | about 71.64 |
| Glycerin | about 7.50 |
| Ethylhexyl Palmitate | about 4.00 |
| Cetearyl Alcohol | about 2.00 |
| Propylene Glycol | about 2.50 |
| Dicetyl Phosphate | about 1.40 |
| Theobroma Grandiflorum Seed Butter | about 2.00 |
| Petrolatum | about 1.00 |
| Dimethicone | about 1.00 |
| A. nidulans Extract | about 1 |
| Arabidopsis Thaliana Extract | about 1 |
| Micrococcus Luteus Lysate | about 1 |
| Lecithin | about 1 |
| Ceteareth-10 Phosphate | about 0.60 |
| Steareth-10 | about 0.86 |
| Hydroxypropyl Bispalmitamide MEA | about 0.50 |
| (Ceramide) | |
| Tocopheryl Acetate | about 1.00 |
| Disodium EDTA | about 0.10 |
| Phenoxyethanol | about 0.80 |
| Sodium Hyaluronate | about 0.10 |
| Sodium Hydroxide | |

In certain embodiments, the invention relates to any one of the aforementioned topical formulations, wherein the topical formulation comprises, consists essentially of, or consists of

| | % by weight of the |
|---|---|
| Ingredient | topical formulation |
| a diluent | from about 45 to about 95 |
| a thickener | from about 0.25 to about 0.8 |
| a humectant or a solvent | from about 4 to about 12 |
| an anti-irritant | from about 0.5 to about 1.5 |
| a DNA repair enzyme | from about 1.5 to about 4.5 |
| a suspending agent | from about 0.5 to about 1.5 |
| an emollient | from about 3 to about 9 |
| a chelating agent | from about 0.05 to about 0.15 |
| a preservative | from about 0.5 to about 1.5 |
| pH Adjuster | |

In certain embodiments, the invention relates to any one of the aforementioned topical formulations, wherein the topical formulation comprises, consists essentially of, or consists of

| | % by weight of the |
|---|---|
| Ingredient | topical formulation |
| Water | from about 45 to about 95 |
| Carbomer 980 | from about 0.1 to about 0.6 |
| Acrylates/C10-C30 Alkyl Acrylate | from about 0.1 to about 0.3 |
| Crosspolymer | |
| Glycerin | from about 1.5 to about 4.5 |
| Propanediol | from about 1.5 to about 4.5 |
| Disodium EDTA | from about 0.05 to about 0.15 |
| Sodium Hyaluronate | from about 1 to about 3 |
| Evodia Rutaecarpia Fruit Extract | from about 0.5 to about 1.5 |
| A. nidulans Extract | from about 0.5 to about 1.5 |
| Arabidopsis Thaliana Extract | from about 0.5 to about 1.5 |
| Micrococcus Luteus Lysate | from about 0.5 to about 1.5 |
| Lecithin | from about 0.5 to about 1.5 |
| Ethylhexyl Isononanoate | from about 2.50 |
| Isononyl Isononanoate | from about 2.50 |
| Butylene Glycol | from about 0.0001 |
| BHT | from about <1% |
| Ergothioneine | from about 0.0001 |
| Santalam Album (Sandalwood) Extract | from about 0.33 |
| Phellodendron Amurense Bark Extract | from about 0.33 |
| Hordeum Distichon (Barley) Extract | from about 0.33 |
| Sodium Hydroxide | |

In certain embodiments, the invention relates to any one of the aforementioned topical formulations, wherein the topical formulation comprises, consists essentially of, or consists of

| | %, by weight of the |
|---|---|
| Ingredient | topical formulation |
| Water | about 85.85 |
| Carbomer 980 | about 0.35 |
| Acrylates/C10-C30 Alkyl Acrylate | about 0.20 |
| Crosspolymer | |
| Glycerin | about 3.00 |
| Propanediol | about 3.00 |
| Disodium EDTA | about 0.10 |
| Sodium Hyaluronate | about 2.00 |
| Evodia Rutaecarpia Fruit Extract | about 1 |
| A. nidulans Extract | about 1 |
| Arabidopsis Thaliana Extract | about 1 |
| Micrococcus Luteus Lysate | about 1 |
| Lecithin | about 1 |
| Ethylhexyl Isononanoate | about 2.50 |
| Isononyl Isononanoate | about 2.50 |
| Butylene Glycol | about 0.0001 |
| Butylated hydroxy toluene (BHT) | about 1 |
| Ergothioneine | about 0.0001 |
| Santalam Album (Sandalwood) Extract | about 0.33 |
| Phellodendron Amurense Bark Extract | about 0.33 |
| Hordeum Distichon (Barley) Extract | about 0.33 |
| Sodium Hydroxide | |

### Exemplary Methods

In certain embodiments, the invention relates to a method of decreasing or preventing UV-induced skin damage, comprising the step of applying to an area of skin an effective amount of any one of the aforementioned topical formulations.

In certain embodiments, the invention relates to any one of the aforementioned methods, wherein said application of the topical formulation is prior to exposure to sunlight.

In certain embodiments, the invention relates to any one of the aforementioned methods, wherein said application of the topical formulation is after exposure to sunlight.

In certain embodiments, the invention relates to a method of decreasing or preventing UV-induced skin damage, comprising the steps of: applying to an area of skin prior to exposure to sunlight an effective amount of any one of the aforementioned topical formulations; and applying to an area of skin after exposure to sunlight an effective amount of any one of the aforementioned topical formulations.

In certain embodiments, the invention relates to a method of decreasing or preventing UV-induced skin damage, comprising the steps of: applying to an area of skin prior to exposure to sunlight an effective amount of a topical formulation comprising, consisting essentially of, or consisting of a photolyase and a dermatologically acceptable carrier or excipient; and applying to the area of skin after exposure to sunlight an effective amount of a topical formulation comprising, consisting essentially of, or consisting of an endonuclease and a dermatologically acceptable carrier or excipient.

In certain embodiments, the invention relates to any one of the aforementioned methods, wherein the photolyase is from A. nidulans.

In certain embodiments, the invention relates to any one of the aforementioned methods, wherein the endonuclease is from M. luteus.

In certain embodiments, the invention relates to any one of the aforementioned methods, wherein the photolyase is from A. nidulans; and the endonuclease is from M. luteus.

In certain embodiments, the invention relates to any one of the aforementioned methods, wherein the topical formulation comprises, consists essentially of, or consists of

| | % by weight of the |
|---|---|
| Ingredient | topical formulation |
| a diluent | from about 35 to about 95 |
| a humectant or a solvent | from about 5 to about 15 |
| a skin conditioning agent or an emollient | from about 4 to about 12 |
| a DNA repair enzyme | from about 1.5 to about 4.5 |
| a suspending agent | from about 0.1 to about 2 |
| an emulsion stabilizer, an emulsifier, or | from about 2.5 to about 7.5 |
| a surfactant | |
| an antioxidant | from about 0.5 to about 1.5 |
| a chelating agent | from about 0.05 to about 0.15 |
| a preservative | from about 0.4 to about 1.2 |
| a pH adjuster | |

In certain embodiments, the invention relates to any one of the aforementioned methods, wherein the formulation comprises, consists essentially of, or consists of

| | % by weight of the |
|---|---|
| Ingredient | topical formulation |
| Water | from about 35 to about 95 |
| Glycerin | from about 3 to about 11 |
| Ethylhexyl Palmitate | from about 2 to about 6 |
| Cetearyl Alcohol | from about 1 to about 3 |
| Propylene Glycol | from about 1 to about 4 |
| Dicetyl Phosphate | from about 0.5 to about 2.5 |
| Theobroma Grandiflorum Seed Butter | from about 1 to about 3 |
| Petrolatum | from about 0.5 to about 1.5 |
| Dimethicone | from about 0.5 to about 1.5 |
| A. nidulans Extract | from about 0.5 to about 1.5 |
| Arabidopsis Thaliana Extract | from about 0.5 to about 1.5 |
| Micrococcus Luteus Lysate | from about 0.5 to about 1.5 |
| Lecithin | from about 0.5 to about 1.5 |
| Ceteareth-10 Phosphate | from about 0.3 to about 0.9 |
| Steareth-10 | from about 0.3 to about 0.9 |
| Hydroxypropyl Bispalmitamide MEA | from about 0.2 to about 0.8 |
| (Ceramide) | |
| Tocopheryl Acetate | from about 0.5 to about 1.5 |
| Disodium EDTA | from about 0.05 to about 0.15 |
| Phenoxyethanol | from about 0.4 to about 1.2 |
| Sodium Hyaluronate | from about 0.05 to about 0.15 |
| Sodium Hydroxide | |

In certain embodiments, the invention relates to any one of the aforementioned methods, wherein the formulation comprises, consists essentially of, or consists of

| | % by weight of the |
|---|---|
| Ingredient | topical formulation |
| Water | about 71.64 |
| Glycerin | about 7.50 |
| Ethylhexyl Palmitate | about 4.00 |
| Cetearyl Alcohol | about 2.00 |
| Propylene Glycol | about 2.50 |
| Dicetyl Phosphate | about 1.40 |
| Theobroma Grandiflorum Seed Butter | about 2.00 |
| Petrolatum | about 1.00 |
| Dimethicone | about 1.00 |
| A. nidulans Extract | about 1 |
| Arabidopsis Thaliana Extract | about 1 |
| Micrococcus Luteus Lysate | about 1 |
| Lecithin | about 1 |
| Ceteareth-10 Phosphate | about 0.60 |
| Steareth-10 | about 0.86 |
| Hydroxypropyl Bispalmitamide MEA | about 0.50 |
| (Ceramide) | |
| Tocopheryl Acetate | about 1.00 |
| Disodium EDTA | about 0.10 |
| Phenoxyethanol | about 0.80 |
| Sodium Hyaluronate | about 0.10 |
| Sodium Hydroxide | |

In certain embodiments, the invention relates to any one of the aforementioned methods, wherein the topical formulation comprises, consists essentially of, or consists of

| | % by weight of the |
|---|---|
| Ingredient | topical formulation |
| a diluent | from about 45 to about 95 |
| a thickener | from about 0.25 to about 0.8 |
| a humectant or a solvent | from about 4 to about 12 |
| an anti-irritant | from about 0.5 to about 1.5 |
| a DNA repair enzyme | from about 1.5 to about 4.5 |
| a suspending agent | from about 0.5 to about 1.5 |
| an emollient | from about 3 to about 9 |
| a chelating agent | from about 0.05 to about 0.15 |
| a preservative | from about 0.5 to about 1.5 |
| pH Adjuster | |

In certain embodiments, the invention relates to any one of the aforementioned methods, wherein the topical formulation comprises, consists essentially of, or consists of

| | % by weight of the |
|---|---|
| Ingredient | topical formulation |
| Water | from about 45 to about 95 |
| Carbomer 980 | from about 0.1 to about 0.6 |
| Acrylates/C10-C30 Alkyl Acrylate | from about 0.1 to about 0.3 |
| Crosspolymer | |
| Glycerin | from about 1.5 to about 4.5 |
| Propanediol | from about 1.5 to about 4.5 |
| Disodium EDTA | from about 0.05 to about 0.15 |
| Sodium Hyaluronate | from about 1 to about 3 |
| Evodia Rutaecarpia Fruit Extract | from about 0.5 to about 1.5 |
| A. nidulans Extract | from about 0.5 to about 1.5 |
| Arabidopsis Thaliana Extract | from about 0.5 to about 1.5 |
| Micrococcus Luteus Lysate | from about 0.5 to about 1.5 |
| Lecithin | from about 0.5 to about 1.5 |
| Ethylhexyl Isononanoate | from about 2.50 |
| Isononyl Isononanoate | from about 2.50 |
| Butylene Glycol | from about 0.0001 |
| BHT | from about <1% |
| Ergothioneine | from about 0.0001 |
| Santalam Album (Sandalwood) Extract | from about 0.33 |
| Phellodendron Amurense Bark Extract | from about 0.33 |
| Hordeum Distichon (Barley) Extract | from about 0.33 |
| Sodium Hydroxide | |

In certain embodiments, the invention relates to any one of the aforementioned methods, wherein the topical formulation comprises, consists essentially of, or consists of

| | %, by weight of the |
|---|---|
| Ingredient | topical formulation |
| Water | about 85.85 |
| Carbomer 980 | about 0.35 |
| Acrylates/C10-C30 Alkyl Acrylate | about 0.20 |
| Crosspolymer | |
| Glycerin | about 3.00 |
| Propanediol | about 3.00 |
| Disodium EDTA | about 0.10 |
| Sodium Hyaluronate | about 2.00 |
| Evodia Rutaecarpia Fruit Extract | about 1 |
| A. nidulans Extract | about 1 |
| Arabidopsis Thaliana Extract | about 1 |
| Micrococcus Luteus Lysate | about 1 |
| Lecithin | about 1 |
| Ethylhexyl Isononanoate | about 2.50 |
| Isononyl Isononanoate | about 2.50 |
| Butylene Glycol | about 0.0001 |
| Butylated hydroxy toluene (BHT) | about 1 |
| Ergothioneine | about 0.0001 |
| Santalam Album (Sandalwood) Extract | about 0.33 |
| Phellodendron Amurense Bark Extract | about 0.33 |
| Hordeum Distichon (Barley) Extract | about 0.33 |
| Sodium Hydroxide | |

### EXEMPLIFICATION

### Clinical Methods

### Subjects

Twelve healthy Caucasian volunteers (6 males and 6 females, age range: 26 to 31 years) with Fitzpatrick skin type I-II were recruited for the study. None of the subjects used any medication or had a history of photodermatosis and skin cancer. In accordance with previous methodology, the study was conducted in winter to minimize the effect of ambient sun exposure. The study protocol was approved by the local Medical Research Ethics Committee and written informed consent was obtained from each subject before participation in the study.

### Test Materials

The SS (sun protection factor [SPF] 50) contained Tinosorb M, 50% solution (4%), Parsol MCX (8%), Tinosorb S (5%), Eusolex 9020 (2%), and Eusolex OCR (1%). The photolyase preparation contained 1% w/w of photolyase derived from the cyanobacterium A. nidulans in a liposomal preparation. The endonuclease preparation contained M. luteus-derived endonuclease incapsulated in liposomes (1% w/w). The vehicle (placebo) was a commercially available moisturizer base.

### Solar Simulator

Solar-simulated radiation was produced by an Oriel solar simulator (Model 81292, L.O.T. Oriel, Leatherhead, UK) containing a 1 kW xenon arc lamp with two dichroic mirrors, a collimator, and a 1-mm WG320 filter. The optical design of this particular solar simulator gives a field of even irradiance (290-400 nm) at the skin surface when positioned 11 cm from the source, of which about 10% is UVB (280-320 nm) and the remainder UVA. The spectral irradiance was measured with an OL754 spectroradiometer (Optronics, Orlando, Fla., USA), calibrated for wavelength and intensity against standard lamps. The spectroradiometer was used to calibrate a handheld IL700 radiometer (International Light, Newburyport, Mass., USA), which was then used to rapidly monitor lamp output on a daily basis.

### Irradiation and Treatment Protocol

Two weeks before the test irradiations, the minimal erythema dose (MED) was determined for each individual for solar-simulated UVR (290-400 nm) and expressed in mJ/cm² by using a light-proof adhesive-backed foil template that were sequentially uncovered to deliver quantities of UVR above and below the expected MED of skin phototype II individuals for solar-simulating UVR. The sites were examined 24 h after irradiation and the MED was determined as the site that showed minimal, uniform perceptible erythema. Before irradiation, six circular areas (10 mm diameter) were marked out on the nonexposed lower back of each participant. One site was left untreated (site 1: negative control), whereas the remaining 5 sites (designated sites 2-6) were exposed to solar-simulated UVR at 3 times the MED on four consecutive days. Site 2 received ssUVR only (site 2: positive control), whereas the following products were applied to sites 3-6, respectively: vehicle (moisturizer base cream; applied both thirty minutes before and immediately after each irradiation; site 3); a traditional sunscreen (SS, SPF 50) thirty minutes before irradiation and a vehicle immediately after irradiation (site 4); a SS thirty minutes before irradiation and an endonuclease preparation immediately after irradiation (site 5); a SS plus photolyase thirty minutes before irradiation and an endonuclease preparation immediately after irradiation (site 6). Twenty-four hours after the last exposure to UVR, skin specimens were obtained through a 4-mm punch biopsy from all sites for molecular analyses.

TABLE 1
Treatment description for the six experimental sites

| | | Solar-simulated |
|---|---|---|
| Site | Condition | ultraviolet radiation |
| 1 | Baseline (reference) | − |
| 2 | Ultraviolet radiation only | + |
| 3 | Vehicle | + |
| 4 | Sunscreen alone before irradiation | + |
| 5 | Sunscreen before irradiation and | + |
| | endonuclease after irradiation | |
| 6 | Sunscreen plus photolyase before | + |
| | irradiation and endonuclease after | |
| | irradiation | |

### DNA Extraction and Measurements of Telomere Length in Skin Biopsies

The skin biopsy specimens were cleaved in half, and one piece was thawed at room temperature, minced, and lysed by three cycles of freezing (in an ethanol-dry-ice bath) and thawing (at 95° C.). Samples were digested for 12 h at 60° C. with proteinase K in 100 mmol/liter Tris-HCl (pH 7.4), 150 mmol/L NaCl, and 10 mmol/L EDTA (pH 8.0). Proteinase K was heat inactivated at 95° C. for 10 min, and homogenates were extracted using the Puregene DNA Isolation kit (Gentra Systems, Minneapolis, Minn., USA). The kit contains two main reagents: cell lysis and protein precipitation solutions. In brief, DNA was extracted from homogenates using a lysis buffer solution and then treated with RNase A. The kit removes proteins using a precipitation solution, followed by 2-propanol to pellet the DNA. Telomere length was measured as abundance of telomeric template (T) vs a single gene copy (S) by quantitative real-time PCR as previously described²⁵ with slight modifications. For the T/S analysis, a 5 μL-aliquot with 20 ng of DNA and 10 μL of master mix were added to each sample well. For each standard curve one reference DNA sample was serially diluted in H₂O by 1.68-fold per dilution to produce five concentrations of DNA ranging from 30 ng to 2 ng in 5 μL. The composition of T and S PCRs were identical except for the oligonucleotide primers. Telomere and single copy gene (36B4) were analyzed in the same plate in order to reduce inter-assay variability. Measurements were performed in triplicate and reported as T/S ratio in respect to a calibrator sample. The same calibrator sample was used in all runs to allow comparison across runs. Every PCR was carried out on a BioRad iQ5 Cycler (BioRad, Hercules, Calif., USA). The PCR protocol for the amplicons begun with a 95° C. incubation for 10 min followed by 30 cycles of 95° C. for 5 sec, 57° C. for 15 sec and 72° C. for 20 sec. The melting curve values for telomere length and single copy gene corresponded to the expected values. The coefficients of variation within duplicates of the telomere and single-gene assay were 2.1% and 1.8%, respectively.

RNA Extraction and Assessment of c-FOS Expression in Skin Biopsies

RNA from skin samples was isolated using the RNeasy Mini Kit (Qiagen, Valencia, Calif., USA) according to the manufacturer's instructions. Integrity of RNA was assessed with agarose gel electrophoresis, and RNA quantity was measured by spectrophotometry. A 1 μg amount of RNA was reverse transcribed using the iScript cDNA Synthesis Kit (BioRad) according to the manufacturer's instructions. cDNA was stored at −20° C. In brief, a 25 μL reaction solution consisted of iQ SYBR Green Supermix (BioRad), forward and reverse primers (final concentration 400 nM each), and cDNA mixture (40 ng). The primers for c-FOS were: forward 5′-TCTCTTACTACCACTCACCC-3′ and reverse 5′TGGAGTGTATCAGTCAGCTC-3′ as described previously.²⁶ To control for variations in RNA quality and quantity, the expression of the gene of interest was normalized to the expression of hypoxanthine phosphoribosyltransferase-1 (HPRT1) as a housekeeping gene. mRNA expression levels were calculated according to the following formula: 2−ΔCT, where ΔCT (sample) was defined as CT (c-FOS)−CT (HPRT1).

### Data Analysis

Statistical analyses were carried out using SPSS for Windows, version 14.0 (SPSS Inc., Chicago, Ill., USA). Because the Shapiro-Wilk test provided evidence that the data were normally distributed, only parametric statistics were used. Continuous variables are presented as means and standard deviations, whereas categorical variables are given as counts. One-way ANOVA followed by Newman-Keuls multiple-comparison post-hoc test was used to analyze intergroup differences. Given the exploratory nature of the study, no Bonferroni correction was used. A probability value <0.05 (two-tailed) was considered statistically significant.

### Clinical Results

The mean MED for solar-simulated UVR was 51±8 mJ/cm². The MEDs were recorded for each individual and the experimental schedule was initiated using the solar simulator based on the initial MED for each individual.

### Telomere Length

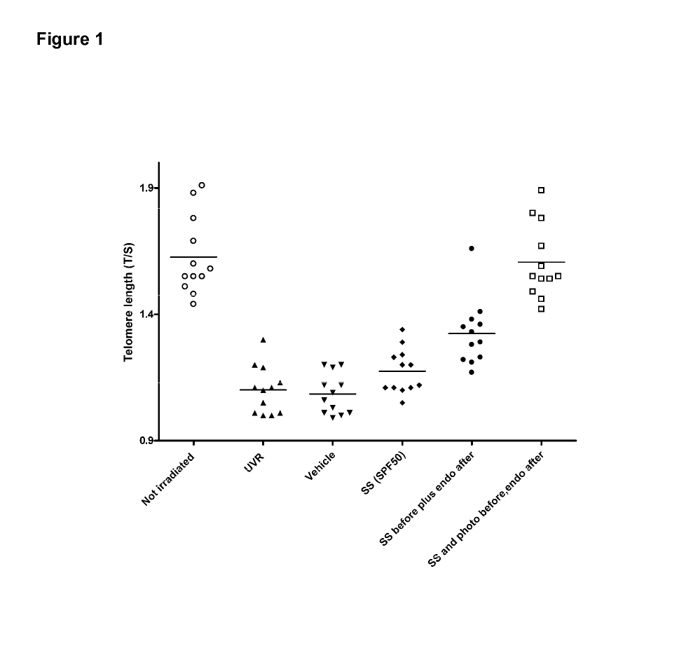

The effect of repeated UVR irradiations on telomere length in human skin in vivo is depicted in FIG. 1. Repeated irradiations were significantly associated with a marked reduction in telomere length both in the UVR only positive control (mean T/S=1.10±0.09, P<0.001) and vehicle (placebo)+UVR sites (mean T/S=1.10±0.10, P<0.001) compared with the nonirradiated site (mean T/S=1.60±0.15). The application of a SS alone before each irradiation was significantly but modestly associated with a slightly longer telomere length in skin biopsies (mean T/S=1.20±0.09, P<0.05) as compared with the non-irradiated skin and in the vehicle (placebo)+UVR site. However, the application of the SS before and endonuclease after each irradiation resulted in a mean T/S of 1.30±0.13 (P<0.01), which indicated that the telomere length was significantly higher than that observed at the site treated by the SS alone. Remarkably, the T/S value of the site treated by SS plus photolyase thirty minutes before each irradiation and endonuclease immediately after irradiation was similar to that found in the non-irradiated skin (1.58±0.13), and was thus higher than that observed with the application of the SS before and endonuclease thereafter each UVR application (P<0.001).

c-FOS Expression

Expression analysis of c-FOS was successful in all of the skin biopsies. As expected (Table 2), repeated irradiations of the human skin significantly increased c-FOS expression in both UVR only positive control and vehicle+UVR sites (P<0.001 at both sites compared with the non-irradiated site, P<0.001). Of note, c-FOS expression in the site treated by both SS plus photolyase thirty minutes before each irradiation and an endonuclease preparation immediately after irradiation was similar to that observed in the non-irradiated site (Table 2). By contrast, c-FOS hyperexpression was significantly reduced but not completely abrogated both by the SS alone and by the combined application of the SS before and the endonuclease preparation after each irradiation.

TABLE 2
c-FOS expression values for the six experimental sites

| | | Solar-simulated | |
|---|---|---|---|
| | | ultraviolet | c-FOS |
| Site | Condition | radiation | expression |
| 1 | Baseline (reference) | − | 0.74 ± 0.19 |
| 2 | Ultraviolet radiation only | + | 1.27 ± 0.22 |
| 3 | Vehicle | + | 1.24 ± 0.33 |
| 4 | Sunscreen alone before irradia- | + | 1.11 ± 0.42 |
| | tion | | |
| 5 | Sunscreen before irradiation and | + | 0.97 ± 0.27 |
| | endonuclease after irradiation | | |
| 6 | Sunscreen plus photolyase before | + | 0.78 ± 0.22 |
| | irradiation and endonuclease | | |
| | after irradiation | | |

Messenger ribonucleic acid expression levels were calculated according to the formula 2−ΔCT, where ΔCT (sample) was defined as CT (c-FOS)−CT (HPRT1).

### REFERENCES CITED

1. Fisher G J, Wang Z Q, Datta S C, Varani J, Kang S, Voorhees J J. Pathophysiology of premature skin aging induced by ultraviolet light. N Engl J Med 1997; 337:1419-1428.
2. Marghoob A A. Skin cancers and their etiologies. Semin Cutan Med Surg 2011; 30:S1-5.
3. Narayanan D L, Saladi R N, Fox J L. Ultraviolet radiation and skin cancer. Int J Dermatol 2010; 49:978-986.
4. Cadet J, Sage E, Douki T. Ultraviolet radiation-mediated damage to cellular DNA. Mutat Res 2005; 571:3-17.
5. Schwarz T, Schwarz A. Molecular mechanisms of ultraviolet radiation-induced immunosuppression. Eur J Cell Biol 2011; 90:560-564.
6. Pfeifer G P, Besaratinia A. UV wavelength-dependent DNA damage and human non-melanoma and melanoma skin cancer. Photochem Photobiol Sci 2012; 11:90-97.
7. Vink A A, Roza L. Biological consequences of cyclobutane pyrimidine dimers. J Photochem Photobiol B 2001; 65:101-104.
8. Durbeej B, Eriksson L A. On the formation of cyclobutane pyrimidine dimers in UV-irradiated DNA: why are thymines more reactive? Photochem Photobiol 2003; 78:159-167.
9. Berardesca E, Bertona M, Altabas K, Altabas V, Emanuele E. Reduced ultraviolet-induced DNA damage and apoptosis in human skin with topical application of a photolyase-containing DNA repair enzyme cream: clues to skin cancer prevention. Mol Med Report 2012; 5:570-574.
10. Stege H, Roza L, Vink A A, Grewe M, Ruzicka T, Grether-Beck S, Krutmann J. Enzyme plus light therapy to repair DNA damage in ultraviolet-B-irradiated human skin. Proc Natl Acad Sci USA 2000; 97:1790-1795.
11. Shiota S, Nakayama H. UV endonuclease of Micrococcus luteus, a cyclobutane pyrimidine dimer-DNA glycosylase/abasic lyase: cloning and characterization of the gene. Proc Natl Acad Sci USA 1997; 94:593-598.
12. Piersen C E, Prince M A, Augustine M L, Dodson M L, Lloyd R S. Purification and cloning of Micrococcus luteus ultraviolet endonuclease, an N-glycosylase/abasic lyase that proceeds via an imino enzyme-DNA intermediate. J Biol Chem 1995; 270:23475-23484.
13. Stege H. Effect of xenogenic repair enzymes on photoimmunology and photocarcinogenesis. J Photochem Photobiol B 2001; 65:105-108.
14. Hofer A, Legat F J, Gruber-Wackernagel A, Quehenberger F, Wolf P. Topical liposomal DNA-repair enzymes in polymorphic light eruption. Photochem Photobiol Sci 2011; 10:1118-1128.
15. Liu Z, Tan C, Guo X, Kao Y T, Li J, Wang L, Sancar A, Zhong D. Dynamics and mechanism of cyclobutane pyrimidine dimer repair by DNA photolyase. Proc Natl Acad Sci USA 2011; 108:14831-6.
16. Buckingham E M, Klingelhutz A J. The role of telomeres in the ageing of human skin. Exp Dermatol 2011; 20:297-302.
17. Rochette P J, Brash D E. Human telomeres are hypersensitive to UV-induced DNA Damage and refractory to repair. PLoS Genet. 2010; 6:e1000926.
18. Gilchrest B A, Eller M S, Yaar M. Telomere-mediated effects on melanogenesis and skin aging. J Investig Dermatol Symp Proc 2009; 14:25-31.
19. Han J, Qureshi A A, Prescott J, Guo Q, Ye L, Hunter D J, De Vivo I. A prospective study of telomere length and the risk of skin cancer. J Invest Dermatol 2009; 129:415-21.
20. Saez E, Rutberg S E, Mueller E, Oppenheim H, Smoluk J, Yuspa S H, Spiegelman B M. c-fos is required for malignant progression of skin tumors. Cell 1995; 82:721-32.
21. Yuspa S H, Greenhalgh D A. Molecular mechanisms of malignant conversion in skin carcinogenesis. Princess Takamatsu Symp 1989; 20:281-288.
22. Batista L F, Artandi S E. Telomere uncapping, chromosomes, and carcinomas. Cancer Cell 2009; 15:455-7.
23. Olson E R, Melton T, Dickinson S E, Dong Z, Alberts D S, Bowden G T. Quercetin potentiates UVB-Induced c-Fos expression: implications for its use as a chemopreventive agent. Cancer Prev Res (Phila) 2010; 3:876-884.
24. Roddey P K, Garmyn M, Park H Y, Bhawan J, Gilchrest B A. Ultraviolet irradiation induces c-fos but not c-Ha-ras proto-oncogene expression in human epidermis. J Invest Dermatol 1994; 102:296-299.
25. Cawthon R M. Telomere measurement by quantitative PCR. Nucleic Acids Res 2002; 30:e47.
26. Walker L M, Preston M R, Magnay J L, Thomas P B, El Haj A J. Nicotinic regulation of c-fos and osteopontin expression in human-derived osteoblast-like cells and human trabecular bone organ culture. Bone 2001; 28:603-608.
27. Mouret S, Bogdanowicz P, Haure M J, Castex-Rizzi N, Cadet J, Favier A, Douki T. Assessment of the photoprotection properties of sunscreens by chromatographic measurement of DNA damage in skin explants. Photochem Photobiol 2011; 87:109-16.
28. Ostoich P, Pottier G, Martien S, Chelli F, Morat L, Ricoul M, Abbadie C, Sabatier L. Characterization of telomere maintenance and chromosomal integrity in human fibroblasts and keratinocytes. Radioprotection 2008; 43:095.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A topical formulation, comprising a photolyase; an endonuclease; and a dermatologically acceptable carrier or excipient.

2. The topical formulation of claim 1, wherein the photolyase is from A. nidulans.

3. The topical formulation of claim 1, wherein the endonuclease is from M. luteus.

4. The topical formulation of claim 1, wherein the photolyase is from A. nidulans; and the endonuclease is from M. luteus.

5. The topical formulation of claim 1, wherein the topical formulation consists essentially of

| | % by weight of the |
|---|---|
| Ingredient | topical formulation |
| a diluent | from about 35 to about 95 |
| a humectant or a solvent | from about 5 to about 15 |
| a skin conditioning agent or an emollient | from about 4 to about 12 |
| a DNA repair enzyme | from about 1.5 to about 4.5 |
| a suspending agent | from about 0.1 to about 2 |
| an emulsion stabilizer, an emulsifier, or | from about 2.5 to about 7.5 |
| a surfactant | |
| an antioxidant | from about 0.5 to about 1.5 |
| a chelating agent | from about 0.05 to about 0.15 |
| a preservative | from about 0.4 to about 1.2 |
| a pH adjuster | |

6. The topical formulation of claim 1, wherein the topical formulation consists essentially of

| | % by weight of the |
|---|---|
| Ingredient | topical formulation |
| Water | from about 35 to about 95 |
| Glycerin | from about 3 to about 11 |
| Ethylhexyl Palmitate | from about 2 to about 6 |
| Cetearyl Alcohol | from about 1 to about 3 |
| Propylene Glycol | from about 1 to about 4 |
| Dicetyl Phosphate | from about 0.5 to about 2.5 |
| Theobroma Grandiflorum Seed Butter | from about 1 to about 3 |
| Petrolatum | from about 0.5 to about 1.5 |
| Dimethicone | from about 0.5 to about 1.5 |
| A. nidulans Extract | from about 0.5 to about 1.5 |
| Arabidopsis Thaliana Extract | from about 0.5 to about 1.5 |
| Micrococcus Luteus Lysate | from about 0.5 to about 1.5 |
| Lecithin | from about 0.5 to about 1.5 |
| Ceteareth-10 Phosphate | from about 0.3 to about 0.9 |
| Steareth-10 | from about 0.3 to about 0.9 |
| Hydroxypropyl Bispalmitamide MEA | from about 0.2 to about 0.8 |
| (Ceramide) | |
| Tocopheryl Acetate | from about 0.5 to about 1.5 |
| Disodium EDTA | from about 0.05 to about 0.15 |
| Phenoxyethanol | from about 0.4 to about 1.2 |
| Sodium Hyaluronate | from about 0.05 to about 0.15 |
| Sodium Hydroxide | |

7. The topical formulation of claim 1, wherein the topical formulation consists essentially of

| | % by weight of |
|---|---|
| Ingredient | topical formulation |
| Water | about 71.64 |
| Glycerin | about 7.50 |
| Ethylhexyl Palmitate | about 4.00 |
| Cetearyl Alcohol | about 2.00 |
| Propylene Glycol | about 2.50 |
| Dicetyl Phosphate | about 1.40 |
| Theobroma Grandiflorum Seed Butter | about 2.00 |
| Petrolatum | about 1.00 |
| Dimethicone | about 1.00 |
| A. nidulans Extract | about 1 |
| Arabidopsis Thaliana Extract | about 1 |
| Micrococcus Luteus Lysate | about 1 |
| Lecithin | about 1 |
| Ceteareth-10 Phosphate | about 0.60 |
| Steareth-10 | about 0.86 |
| Hydroxypropyl Bispalmitamide MEA | about 0.50 |
| (Ceramide) | |
| Tocopheryl Acetate | about 1.00 |
| Disodium EDTA | about 0.10 |
| Phenoxyethanol | about 0.80 |
| Sodium Hyaluronate | about 0.10 |
| Sodium Hydroxide | |

8. The topical formulation of claim 1, wherein the topical formulation consists essentially of

| | % by weight of the |
|---|---|
| Ingredient | topical formulation |
| a diluent | from about 45 to about 95 |
| a thickener | from about 0.25 to about 0.8 |
| a humectant or a solvent | from about 4 to about 12 |
| an anti-irritant | from about 0.5 to about 1.5 |
| a DNA repair enzyme | from about 1.5 to about 4.5 |
| a suspending agent | from about 0.5 to about 1.5 |
| an emollient | from about 3 to about 9 |
| a chelating agent | from about 0.05 to about 0.15 |
| a preservative | from about 0.5 to about 1.5 |
| pH Adjuster | |

9. The topical formulation of claim 1, wherein the topical formulation consists essentially of

| | % by weight of the |
|---|---|
| Ingredient | topical formulation |
| Water | from about 45 to about 95 |
| Carbomer 980 | from about 0.1 to about 0.6 |
| Acrylates/C10-C30 Alkyl Acrylate | from about 0.1 to about 0.3 |
| Crosspolymer | |
| Glycerin | from about 1.5 to about 4.5 |
| Propanediol | from about 1.5 to about 4.5 |
| Disodium EDTA | from about 0.05 to about 0.15 |
| Sodium Hyaluronate | from about 1 to about 3 |
| Evodia Rutaecarpia Fruit Extract | from about 0.5 to about 1.5 |
| A. nidulans Extract | from about 0.5 to about 1.5 |
| Arabidopsis Thaliana Extract | from about 0.5 to about 1.5 |
| Micrococcus Luteus Lysate | from about 0.5 to about 1.5 |
| Lecithin | from about 0.5 to about 1.5 |
| Ethylhexyl Isononanoate | from about 2.50 |
| Isononyl Isononanoate | from about 2.50 |
| Butylene Glycol | from about 0.0001 |
| BHT | from about <1% |
| Ergothioneine | from about 0.0001 |
| Santalam Album (Sandalwood) Extract | from about 0.33 |
| Phellodendron Amurense Bark Extract | from about 0.33 |
| Hordeum Distichon (Barley) Extract | from about 0.33 |
| Sodium Hydroxide | |

10. The topical formulation of claim 1, wherein the topical formulation consists essentially of

| | %, by weight of the |
|---|---|
| Ingredient | topical formulation |
| Water | about 85.85 |
| Carbomer 980 | about 0.35 |
| Acrylates/C10-C30 Alkyl Acrylate | about 0.20 |
| Crosspolymer | |
| Glycerin | about 3.00 |
| Propanediol | about 3.00 |
| Disodium EDTA | about 0.10 |
| Sodium Hyaluronate | about 2.00 |
| Evodia Rutaecarpia Fruit Extract | about 1 |
| A. nidulans Extract | about 1 |
| Arabidopsis Thaliana Extract | about 1 |
| Micrococcus Luteus Lysate | about 1 |
| Lecithin | about 1 |
| Ethylhexyl Isononanoate | about 2.50 |
| Isononyl Isononanoate | about 2.50 |
| Butylene Glycol | about 0.0001 |
| Butylated hydroxy toluene (BHT) | about 1 |
| Ergothioneine | about 0.0001 |
| Santalam Album (Sandalwood) Extract | about 0.33 |
| Phellodendron Amurense Bark Extract | about 0.33 |
| Hordeum Distichon (Barley) Extract | about 0.33 |
| Sodium Hydroxide | |

11. A method of decreasing or preventing UV-induced skin damage, comprising the step of applying to an area of skin an effective amount of a topical formulation comprising a photolyase; an endonuclease; and a dermatologically acceptable carrier or excipient.

12. The method of claim 11, wherein the photolyase is from A. nidulans.

13. The method of claim 11, wherein the endonuclease is from M. luteus.

14. The method of claim 11, wherein the photolyase is from A. nidulans; and the endonuclease is from M. luteus.

15. The method of claim 11, wherein the topical formulation consists essentially of

| | % by weight of the |
|---|---|
| Ingredient | topical formulation |
| a diluent | from about 35 to about 95 |
| a humectant or a solvent | from about 5 to about 15 |
| a skin conditioning agent or an emollient | from about 4 to about 12 |
| a DNA repair enzyme | from about 1.5 to about 4.5 |
| a suspending agent | from about 0.1 to about 2 |
| an emulsion stabilizer, an emulsifier, or | from about 2.5 to about 7.5 |
| a surfactant | |
| an antioxidant | from about 0.5 to about 1.5 |
| a chelating agent | from about 0.05 to about 0.15 |
| a preservative | from about 0.4 to about 1.2 |
| a pH adjuster | |

16. The method of claim 11, wherein the topical formulation consists essentially of

| | % by weight of the |
|---|---|
| Ingredient | topical formulation |
| Water | from about 35 to about 95 |
| Glycerin | from about 3 to about 11 |
| Ethylhexyl Palmitate | from about 2 to about 6 |
| Cetearyl Alcohol | from about 1 to about 3 |
| Propylene Glycol | from about 1 to about 4 |
| Dicetyl Phosphate | from about 0.5 to about 2.5 |
| Theobroma Grandiflorum Seed Butter | from about 1 to about 3 |
| Petrolatum | from about 0.5 to about 1.5 |
| Dimethicone | from about 0.5 to about 1.5 |
| A. nidulans Extract | from about 0.5 to about 1.5 |
| Arabidopsis Thaliana Extract | from about 0.5 to about 1.5 |
| Micrococcus Luteus Lysate | from about 0.5 to about 1.5 |
| Lecithin | from about 0.5 to about 1.5 |
| Ceteareth-10 Phosphate | from about 0.3 to about 0.9 |
| Steareth-10 | from about 0.3 to about 0.9 |
| Hydroxypropyl Bispalmitamide MEA | from about 0.2 to about 0.8 |
| (Ceramide) | |
| Tocopheryl Acetate | from about 0.5 to about 1.5 |
| Disodium EDTA | from about 0.05 to about 0.15 |
| Phenoxyethanol | from about 0.4 to about 1.2 |
| Sodium Hyaluronate | from about 0.05 to about 0.15 |
| Sodium Hydroxide | |

17. The method of claim 11, wherein the topical formulation consists essentially of

| | % by weight of the |
|---|---|
| Ingredient | topical formulation |
| Water | about 71.64 |
| Glycerin | about 7.50 |
| Ethylhexyl Palmitate | about 4.00 |
| Cetearyl Alcohol | about 2.00 |
| Propylene Glycol | about 2.50 |
| Dicetyl Phosphate | about 1.40 |
| Theobroma Grandiflorum Seed Butter | about 2.00 |
| Petrolatum | about 1.00 |
| Dimethicone | about 1.00 |
| A. nidulans Extract | about 1 |
| Arabidopsis Thaliana Extract | about 1 |
| Micrococcus Luteus Lysate | about 1 |
| Lecithin | about 1 |
| Ceteareth-10 Phosphate | about 0.60 |
| Steareth-10 | about 0.86 |
| Hydroxypropyl Bispalmitamide MEA | about 0.50 |
| (Ceramide) | |
| Tocopheryl Acetate | about 1.00 |
| Disodium EDTA | about 0.10 |
| Phenoxyethanol | about 0.80 |
| Sodium Hyaluronate | about 0.10 |
| Sodium Hydroxide | |

18. The method of claim 11, wherein the topical formulation consists essentially of

| | % by weight of the |
|---|---|
| Ingredient | topical formulation |
| a diluent | from about 45 to about 95 |
| a thickener | from about 0.25 to about 0.8 |
| a humectant or a solvent | from about 4 to about 12 |
| an anti-irritant | from about 0.5 to about 1.5 |
| a DNA repair enzyme | from about 1.5 to about 4.5 |
| a suspending agent | from about 0.5 to about 1.5 |
| an emollient | from about 3 to about 9 |
| a chelating agent | from about 0.05 to about 0.15 |
| a preservative | from about 0.5 to about 1.5 |
| pH Adjuster | |

19. The method of claim 11, wherein the topical formulation consists essentially of

| | % by weight of the |
|---|---|
| Ingredient | topical formulation |
| Water | from about 45 to about 95 |
| Carbomer 980 | from about 0.1 to about 0.6 |
| Acrylates/C10-C30 Alkyl Acrylate | from about 0.1 to about 0.3 |
| Crosspolymer | |
| Glycerin | from about 1.5 to about 4.5 |
| Propanediol | from about 1.5 to about 4.5 |
| Disodium EDTA | from about 0.05 to about 0.15 |
| Sodium Hyaluronate | from about 1 to about 3 |
| Evodia Rutaecarpia Fruit Extract | from about 0.5 to about 1.5 |
| A. nidulans Extract | from about 0.5 to about 1.5 |
| Arabidopsis Thaliana Extract | from about 0.5 to about 1.5 |
| Micrococcus Luteus Lysate | from about 0.5 to about 1.5 |
| Lecithin | from about 0.5 to about 1.5 |
| Ethylhexyl Isononanoate | from about 2.50 |
| Isononyl Isononanoate | from about 2.50 |
| Butylene Glycol | from about 0.0001 |
| BHT | from about <1% |
| Ergothioneine | from about 0.0001 |
| Santalam Album (Sandalwood) Extract | from about 0.33 |
| Phellodendron Amurense Bark Extract | from about 0.33 |
| Hordeum Distichon (Barley) Extract | from about 0.33 |
| Sodium Hydroxide | |

20. The method of claim 11, wherein the topical formulation consists essentially of

| | %, by weight of the |
|---|---|
| Ingredient | topical formulation |
| Water | about 85.85 |
| Carbomer 980 | about 0.35 |
| Acrylates/C10-C30 Alkyl Acrylate | about 0.20 |
| Crosspolymer | |
| Glycerin | about 3.00 |
| Propanediol | about 3.00 |
| Disodium EDTA | about 0.10 |
| Sodium Hyaluronate | about 2.00 |
| Evodia Rutaecarpia Fruit Extract | about 1 |
| A. nidulans Extract | about 1 |
| Arabidopsis Thaliana Extract | about 1 |
| Micrococcus Luteus Lysate | about 1 |
| Lecithin | about 1 |
| Ethylhexyl Isononanoate | about 2.50 |
| Isononyl Isononanoate | about 2.50 |
| Butylene Glycol | about 0.0001 |
| Butylated hydroxy toluene (BHT) | about 1 |
| Ergothioneine | about 0.0001 |
| Santalam Album (Sandalwood) Extract | about 0.33 |
| Phellodendron Amurense Bark Extract | about 0.33 |
| Hordeum Distichon (Barley) Extract | about 0.33 |
| Sodium Hydroxide | |
